(19) United States
(12) Patent Application Publication
(10) Pub. No.: US 2006/0140893 A1
(43) Pub. Date: Jun. 29, 2006

(54) Assay of hair proteins

(75) Inventors: Marine Peron, Paris (FR); Arnaud Lagarde, Gainneville (FR); Joël Bover, Le Thillay (FR)

Correspondence Address: OLIFF & BERRIDGE, PLC, P.O. BOX 19928, ALEXANDRIA, VA, 22320, US

(73) Assignee: L'OREAL, Paris (FR)

(21) Appl. No.: 11/302,214

(22) Filed: Dec. 14, 2005

Related U.S. Application Data
(60) Provisional application No. 60/654,522, filed on Feb. 18, 2005.

(30) Foreign Application Priority Data
Dec. 14, 2004 (FR) 04 52979

(51) Int. Cl. G01N 33/00

(52) U.S. Cl. 424/70.2; 436/86

(57) ABSTRACT

A process for detecting previous treatment of keratinous fibers with alkaline technology for hair straightening or smoothing includes: bringing the keratinous fibers into contact with an extraction solution under conditions effective to cause dissolution of keratin proteins; and conducting a qualitative and/or quantitative assay of the keratin proteins dissolved in the extraction solution. The extraction solution may include a mixture of at least one of urea, thiourea and derivatives thereof with at least one reducing agent, and the at least one reducing agent may be free from thiourea. A kit for performing such a process is also provided.

### CROSS-REFERENCE TO RELATED APPLICATIONS

This non provisional application claims the benefit of French Application No. 04 52979 filed on Dec. 14, 2004 and U.S. Provisional Application No. 60/654,522 filed on Feb. 18, 2005.

### BACKGROUND

A subject-matter of the present invention is a process for rapidly detecting, in keratinous fibers, a previous treatment, in particular of hair straightening or smoothing type, by alkaline technology. It is additionally targeted at providing a kit of use in implementing such a process.

The term “keratinous fibers” is understood to mean, according to the invention, fibers of human or animal origin, such as hairs, including those not on the head, eyelashes, wool, angora, cashmere or fur. Although the invention is not limited to specific keratinous fibers, reference will nevertheless be made more particularly to hairs.

The hair is 85-90% composed of proteins. These proteins belong to two main families: intermediate filaments or alternatively keratin and KAPs (Keratin Associated Proteins). In its entirety, the protein composition of the hair is rich in cysteine in its dimeric form: cystine.

The properties of shaping the hair result in particular from its protein architecture.

Thus, in order to imprint a permanent deformation on the hair, recourse is generally had to actions of chemical cosmetology which occur on the polypeptide chains of the proteins of the hair. These chains comprise sulphur-comprising amino acids, including cysteine in its dimeric form (disulphide): cystine (approximately 15% in g/100 g of total amino acids). These disulphide bridges can be reduced to thiols and then reoxidized, after an imposed deformation, in order to impart stiffness to this deformation. This reaction constitutes one of the cosmetic bases for permanent modifications to the shape of hairs.

According to a first technique, the disulphide bridge is temporarily opened by virtue of the action of a reducing agent, generally a sulphite in the alkaline salt form or a thiol, such as thioglycolic acid. This cleavage action has the effect of severing the disulphide bridges, thus providing the polypeptide chains with a plasticity and an ability to move with respect to one another. The bridges are subsequently reformed at other points, after the desired deformation, by setting with an oxidizing agent.

A second technique consists in carrying out a “lanthionization” operation using a composition comprising a base belonging to the family of the hydroxides. It results in the replacement of a portion of the cystines by lanthionines (monosulphide bonds —CH₂—S—CH₂—). This lanthionization operation involves, in a first step, a β-elimination reaction on the cystine, using a hydroxide ion, and then the reaction of the dihydroalanine thus obtained with a thiol group. In comparison with the first technique, which employs a reducing agent, this lanthionization technique does not require a setting stage. The formation of the lanthionine bridges is irreversible. This technique thus makes it possible to obtain, in a single stage, the straightening or the smoothing of keratinous fibers.

Consequently, the two abovementioned techniques, although leading to the same result namely a permanent deformability, affect the structure of the hair in a different way. In particular, an expert in the hair field knows that the causticity of the alkaline derivatives employed according to the second technique is capable, in some cases, of significantly affecting the state of the hair, in particular rendering it much more brittle. This brittleness is accompanied by a loss in substances and can extend as far as the breaking of the hairs, in particular if they are exposed to consecutive treatments of this type.

Methods for characterizing a previous alkaline hair straightening already exist, in particular by the detection of the presence of lanthionine in the keratinous fibers. This detection is carried out either by assaying the lanthionine after acid hydrolysis and analysis of the amino acids or by the Raman confocal spectroscopy analysis.

Likewise, several methods for collecting proteins have already been provided. Thus, in order to extract cortical proteins, it is known that it is possible to release the proteins of the hair in an aqueous urea solution and then to cut the disulphide bridges with 2-mercaptoethanol or thioglycolic acid (MacLaren. J. A. and Kilpatrick D. J., Aust. J. Biol. Sci., 21, 805-813 [1968]). More recently, provision has been made, in the document JP 2002-114798 A, to combine specific urea compounds in the presence of a reducing agent. The collecting method more particularly described is targeted at eluting and collecting the keratin proteins of the microfibrils and of the matrix constituting the cortical part of the hair by treating these with a mixture of urea and of thiourea, in a ratio of between 5/1 and 1/2, and collecting, from the elution residue, the cuticular part maintained in its shape.

It is clear that these combined techniques, although very reliable in terms of diagnosis, prove to be very restricting in terms of implementation and therefore expensive.

There thus today remains a need for the expert to have available a reliable and rapid means not requiring sophisticated scientific equipment for identifying the “history” of a hair, so as to assess as best as possible the compatibility of consecutive treatment(s) with its state, in particular protein state.

Within the meaning of the invention, the protein state of keratinous fibers is intended to denote their protein concentration according to a qualitative and nonquantitative scale, in other words their protein richness.

### SUMMARY

Unexpectedly, the inventors have found that a test based on the dissolution of the keratin proteins but, on the other hand, not in any way requiring the consecutive isolation of the latter for the purpose of their characterization makes it possible to efficiently distinguish a previous hair straightening by alkaline technology from other treatments, for example of dyeing and/or thioglycolic hair straightening types.

The present invention is targeted specifically at providing a useful and rapid process for detecting whether a keratinous fiber has already been exposed to an alkaline treatment of hair straightening type by alkaline technology, whatever the degree of sensitization of this keratinous fiber.

Conventionally, a hair described as “sensitized” is a hair which has been subjected to at least one oxidation treatment, for example during a dyeing or bleaching, in contrast to a natural hair.

More specifically, the present invention is targeted, according to a first aspect, at a process of use in detecting, in keratinous fiber(s), a previous treatment by alkaline technology, in particular of hair straightening or smoothing type, comprising at least bringing the said keratinous fibers into contact with an “extraction” solution comprising at least urea, thiourea or one of their derivatives, as a mixture with at least one reducing agent other than thiourea, under conditions which are effective in dissolving keratinous proteins, and the qualitative and/or quantitative assaying of the proteins in the said extraction solution.

The present invention is targeted, according to another of its aspects, at a kit of use in characterizing the protein state of keratinous fiber(s) and in particular detecting, in keratinous fiber(s), a previous treatment by alkaline technology, the said kit comprising at least:

one solution comprising urea, thiourea or one of their derivatives,

at least one reducing agent other than thiourea,

at least one compound of use as colorimetric indicator for qualitatively and/or quantitatively assaying keratin proteins.

These and other optional features and possible advantages of various aspects of this invention are described in, or are apparent from, the following detailed description of exemplary embodiments of systems and methods which implement this invention.

### BRIEF DESCRIPTION OF THE DRAWINGS

Various exemplary embodiments of the invention will be described in detail with reference to the following figures, wherein:

FIG. 1 diagrammatically represents, in perspective, a packaging device for a kit in accordance with an implementational example of the invention;

FIG. 2 represents an exemplary kit in accordance with the invention;

FIG. 3 diagrammatically represents a heating device which can be used to bring the small lock and the extraction solution to a predefined temperature; and

FIG. 4 is a diagrammatic view in axial cross section of an alternative embodiment of a packaging device which makes it possible to carry out the mixing of the extraction solution and of the reducing agent at the time of use.

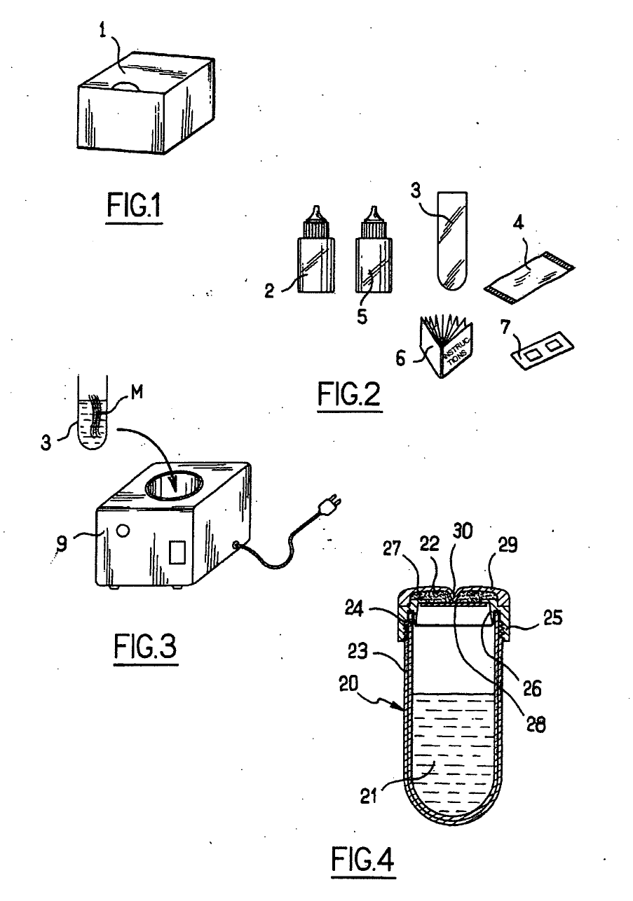

### DETAILED DESCRIPTION OF EMBODIMENTS

Within the meaning of the invention, the expression “qualitative assaying of proteins” denotes an assaying targeted at characterizing the relative abundance of the proteins without precisely evaluating their concentration. In other words, the assaying allows its user to estimate the protein richness of the extraction solution.

A quantitative assaying is targeted, on the other hand, at determining the precise concentration of proteins in the extraction solution, as illustrated in the examples presented below.

Advantageously, the process according to the invention makes it possible to characterize the existence of a previous treatment by alkaline hair straightening, whatever the sensitization treatment or treatments to which the hair under consideration has otherwise been subjected, previously and/or subsequently. The process according to the invention proves to be effective on hairs of any nature, namely natural or sensitized.

Solution for Extraction of Keratin Proteins

As specified above, the mixture employed for the extraction of the proteins is a solution comprising at least urea, thiourea or one of their derivatives and a reducing agent other than thiourea.

Within the meaning of the invention, thiourea or urea derivatives refer to their derivatives of N-alkylated and N-hydroxyalkylated type. These alkyl units can be C₁ to C₁₀ and preferably C₁ to C₄ units and, if appropriate, can be substituted, with the proviso that these substituents are not capable of interfering with the reactivity of the urea or thiourea.

The solution intended to react with regard to keratin proteins generally comprises a mixture of urea and of thiourea or of one of their derivatives in a urea/thiourea ratio varying from 5/1 to 1/2, in particular from 4/1 to 2/1.

More particularly, such a mixture is generally present in a proportion of 20 to 80% by weight, in particular of 30 to 70% by weight, with respect to the total weight of the extraction solution.

These two compounds are used in combination with an effective amount of at least one reducing agent other than thiourea.

Within the meaning of the invention, a reducing agent denotes a compound capable of cutting the disulphide bridges in keratinous fibers and more particularly for the purpose of reducing them.

Derivatives of thiol, phosphine and sulphite type are thus suitable in particular.

According to a specific embodiment of the invention, the reducing agent is a reducing agent of thiol type.

Such agents are chosen in particular from dithiothreitol, thioglycolic acid or thiolactic acid and their ester and amide derivatives, in particular glyceryl monothioglycolate, cysteamine and its C₁-C₄ acylated derivatives, such as N-acetylcysteamine or N-propionylcysteamine, cysteine, N-acetylcysteine, thiomalic acid, pantetheine, 2,3-dimercaptosuccinic acid, sulphites or bisulphites of an alkali metal or alkaline earth metal, N-(mercaptoalkyl)-co-hydroxyalkylamides, such as those disclosed in Patent Application EP-A-354 835, N-mono- or N,N-dialkyl-4-mercaptobutyramides, such as those disclosed in Patent Application EP-A-368 763, aminomercaptoalkylamides, such as those disclosed in Patent Application EP-A-432 000, derivatives of N-(mercaptoalkyl)succinamic acids and N-(mercaptoalkyl)succinimides, such as those disclosed in Patent Application EP-A-465 342, (alkylamino)mercaptoalkylamides, such as those disclosed in Patent Application EP-A-514 282, the azeotropic mixture of 2-hydroxypropyl thioglycolate and of 2-hydroxy-1-methylethyl thioglycolate, such as those disclosed in Patent Application FR-A-2 679 448, mercaptoalkylaminoamides, such as those disclosed in Patent Application FR-A-2 692 481, N-mercaptoalkylalkanediamides, such as those disclosed in Patent Application EP-A-653 202, and formamidinesulphinic acid derivatives, such as those disclosed in Application PCT/US01/43124, filed by the Applicant Company.

This reducing agent is, of course, used at a concentration sufficient to produce the effect desired, namely the reduction of the disulphide bridges. For obvious reasons, this amount is capable of significantly varying according to the nature of the reducing agent under consideration.

In particular, this concentration can be of the order of 0.01 to 2M, in particular of 0.03 to 0.5M and more particularly of 0.04 to 0.1M.

2-Mercaptoethanol, dithiothreitol (DTT), thioglycolic acid, one of their derivatives or their mixtures is more particularly suitable as a reducing agent.

It is also possible to envisage combining this reducing agent with one or more compounds known for their ability to strengthen its effectiveness. By way of illustration, such compounds can be provided in the form of an SiO₂/PDMS (polydimethylsiloxane) mixture, dimethylisosorbitol, pyrrolidone, N-alkylpyrrolidone, thiamorpholinone, alkylene glycol alkyl ethers, dialkylene glycol alkyl ethers, C₃-C₆ alkanediol alkyl ethers or their mixtures.

Such compounds can be present in a proportion of 0.001 to 10% by weight with respect to the weight of the composition.

In addition to urea, thiourea and the reducing compound, the solution intended to dissolve keratin proteins can also comprise a surface-active agent of nonionic, anionic, cationic or amphoteric type. They can in particular be alkyl sulphates, alkylbenzenesulphonates, alkyl ether sulphates, alkylsulphonates, alkyl betaines, oxyalkylenated alkylphenols, fatty acid alkanolamides, oxyalkylenated fatty acid esters, and also oxyalkylenated fatty alcohols and alkylpolyglucosides.

The extraction solution can also comprise one or more acidifying or basifying agents and in particular TRIS (TRIS[hydroxymethyl]aminomethane).

Of course, these agents are chosen so as not to prejudice the reactivity desired with regard to keratin proteins.

As specified above, the process according to the invention involves bringing keratinous fibers into contact with an extraction solution in accordance with the invention.

Generally, a sample of small lock type corresponding to approximately ten hairs is sufficient for carrying out the process in accordance with the invention.

More specifically, the ratio by weight of the keratinous fibers to be analysed to the extraction solution varies from 0.2 to 100 mg/ml, in particular from 1 to 50 mg/ml, and is more particularly of the order of 10 mg/ml.

The amount of keratinous fibers is generally of the order of 50 mg. With regard to the volume of the extraction solution, it is generally of the order of 5 ml.

The small lock of keratinous fibers is brought into contact with the extraction solution, generally by directly immersing in a container, for example of tube or cell type, containing the said solution. These keratinous fibers can advantageously be cut up beforehand to give sections with sizes reduced to the scale of 2 to 3 mm, for example.

This small lock is generally taken by cutting from the head of hair for which it is desired to characterize a previous hair straightening treatment by alkaline technology.

The inventors have demonstrated that the reactivity of the extraction solution is significantly increased if the reaction for dissolving the proteins is carried out under hot conditions, that is to say at a temperature of greater than 50° C.

Consequently, for reasons of speed, the solution comprising the small lock is advantageously heated to a temperature of greater than 50° C., more particularly of greater than 70° C. and in particular of greater than or equal to 100° C. It is understood that this temperature has to be compatible with the hair and must not destroy it.

This heating can be applied by any conventional means. It can thus be heating of waterbath, bottle warmer, microwave and/or hairdryer type.

Of course, the time necessary for the extraction can vary significantly from a minute, indeed even a second, to several hours, depending on the conditions selected, in particular in terms of temperature, for carrying it out.

Generally, the extraction of the proteins is obtained in a time period of the order of 15 minutes, when the reaction is carried out at 100° C., against a time period of 18 hours for a reaction carried out at 40° C.

Furthermore, this heating can be carried out with concomitant stirring of the container containing the keratinous fibers and the extraction solution.

The measurement or evaluation of the relative abundance of the dissolved proteins in the extraction solution can, of course, be carried out using various methods.

It is thus possible to envisage characterizing the abundance of proteins in the solution by an assay method of physical type, such as, for example, turbidity light scattering, of chemical or physicochemical type or alternatively of immunological type.

However, insofar as this test is more particularly intended for styling professionals, it is advantageous to combine this extraction method with a form of detection by colorimetry, in particular based on the Bradford or Lowry method. Such tests are commonly used for assaying proteins.

According to this alternative, all or part of the treatment solution, capable of comprising proteins in a dissolved form, can be brought into contact with an effective amount of a compound, also referred to below as “calorimetric indicator”, capable of interacting with these proteins and of revealing this interaction according to a calorimetric form. It can be either an appearance of color or the modification, indeed even disappearance, of its starting color.

This operation in which the extraction solution is brought into contact with a calorimetric indicator can be carried out in particular by introducing a predetermined sample of the said solution into a solution comprising the colorimetric indicator. The operation in which the calorimetric indicator is brought into contact with all or part of the extraction solution may require the preliminary dilution of this extraction solution. This option, of course, depends on the nature of the calorimetric indicator and is generally stipulated in the procedure for use of the colorimetric indicator.

It is also possible to envisage an alternative embodiment according to which the colorimetric indicator is fixed, in particular by adsorption, to a solid support, for example of narrow strip type, such as, for example, pH paper. The operation of bringing into contact is then carried out by impregnation of the support with all or part of the extraction solution.

The intensity of the color obtained on conclusion of the contacting operation has the additional advantage of being able to be, at the same time, an indicator of the concentration of proteins, with the proviso of having available, with the colored indicator selected, a corresponding calibration.

According to a specific embodiment of the invention, the indicator is based on an acidic solution of Coomassie Blue G-250 capable of changing in terms of colored hues within a coloration range varying from 465 μm to 585 μm when it is combined with proteins.

Mention may in particular be made, by way of illustration of a commercial colorimetric test suitable for the invention, of that distributed by Bio-Rad under the name of Bio-Rad Protein Assay@, a commercial kit for assaying proteins based on the Bradford method.

For the reasons mentioned above, this form of detection of colorimetric type is particularly advantageous in terms of ease of handling and of speed of reading.

It can additionally be desirable to have available, at the same time, a calorimetric control which makes it possible to describe the relative abundance of proteins. This control can be represented by a mixture of keratin proteins of predetermined concentration and in a purified form, in particular extracted from natural hair. However, in the absence of such a reference, it is possible to use any other protein as relative standard. For obvious reasons, it is advantageous to favour the choice of a reference protein which will give an intensity of color similar to that of the protein to be assayed.

Proteins of bovine serum albumin or bovine γ-albumin type, which are proteins available commercially in purified forms, are thus suitable in particular as standard proteins.

As emerges from the examples represented below, the detection of a previous alkaline treatment for hair straightening or smoothing is characterized by the presence of a very reduced, indeed zero, amount of proteins in dissolved form. On the other hand, in the absence of such a previous treatment, the process in accordance with the invention makes it possible to characterize, indeed even to quantify, the presence of a significant amount of keratin proteins in the extraction solution. This presence can be reflected, in the case of a form of detection by colorimetry, by the display of a color, the intensity of which is proportional to the amount of dissolved proteins.

The invention also relates to a kit of use in characterizing the protein state of keratinous fibers and in particular of use in characterizing a previous treatment by alkaline technology for hair straightening or smoothing, comprising at least:

one solution comprising at least urea and thiourea or one of their derivatives,

at least one reducing agent other than thiourea, and

at least one compound of use as calorimetric indicator for qualitatively and/or quantitatively assaying the keratin proteins extracted.

The combined components constituting the solution intended for the extraction of the proteins can be packaged according to a first alternative form in the same container or, according to a second alternative form, in separate containers.

According to this second alternative, the solution of thiourea and of urea, on the one hand, and the reducing agent, on the other hand, are packaged separately. In this case, the containers which are respectively dedicated to them can be conformed so as to be suited to mixing of all their components prior to carrying out the test. For example, it is possible to envisage combining these two containers so as to form a single container at the time of carrying out the test.

According to a first embodiment, the container containing the solution based on urea, on thiourea and, if appropriate, the reducing agent is conformed in order to be suitable for the immersion of keratinous fibers. In this case, it can be equipped with a seal of removable cap type intended to be removed at the time of carrying out the test.

Another alternative form may involve introducing and bringing into contact the sample of keratinous fibers to be characterized, not into the mixture formed beforehand of the two components of the extraction solution but with only one of the components, namely the solution comprising at least the urea and the thiourea or the reducing agent. According to this alternative, the second component is subsequently combined with the preliminary mixture of the fibers with the first component.

According to another embodiment, the kit can additionally comprise a separate container intended to receive the solution of urea and of thiourea, the reducing agent and the keratinous fibers in a non-specific order.

Whatever the embodiment, the container dedicated to the immersion of the small lock during the progression of the test is compatible with heating to a temperature at least equal to 50° C., preferably of greater than 70° C., indeed even of the order of 100° C. It can, for example, be conformed for heating of bottle warmer type.

As regards the calorimetric indicator, it is generally packaged in solution in a container which is specific to it.

All the containers constituting the kit are sealed during their packaging. The forms of sealing are adjusted so as to be favourable to easy opening at the time of carrying out the test and, if appropriate, to controlled release of their content by adjusting a dropper nozzle, for example. The latter option is advantageous in particular for the stage of detection by colorimetry.

In addition, the kit according to the invention advantageously comprises operating instructions in which the user is informed of the instructions to be followed in carrying out the test.

Generally, the procedure to be followed can involve the following stages:

bringing keratinous fibers, more particularly a small lock of hairs which have generally been cut up beforehand in the form of sections with a length of 2 to 3 mm, into contact with the solution of urea and of thiourea and the reducing agent, these two components of the extraction solution having been mixed beforehand or being mixed in the presence of the said keratinous fibers,

if necessary, exposing the container containing the above mixture to a source of heat, if appropriate with stirring, so as to bring the extraction solution to a temperature of greater than 50° C., indeed even 100° C., for a predetermined time period, for example approximately 15 minutes for heating at 100° C.,

if necessary, diluting the extraction solution on conclusion of this heating stage,

introducing a given amount of this solution into the container containing the colorimetric indicator or, conversely, a given amount of the colorimetric indicator into the said extraction solution, and

reading the result while referring, if appropriate, to a calorimetric control.

This colorimetric control may be provided in the kit, indeed even may be presented on the packaging of the kit.

It is also possible to envisage providing, with the kit, a color chart which makes it possible to qualitatively and/or quantitatively assay the proteins extracted as a function of the intensity of color obtained on conclusion of the test.

The kit according to the invention can also comprise reference keratinous fibers for guiding the user on the size of the sample to be taken of the keratinous fibers and in particular of the head of hair to be tested.

A kit which makes it possible to characterize a previous hair treatment can be provided, for example to the user, in a box 1 comprising, as may be seen in FIG. 2, a container 2 containing a solution of urea and of thiourea, a tube 3 or other container intended to receive the small lock and the extraction solution, a sachet 4 or other packaging device comprising the reducing agent, a container 5 containing the colorimetric indicator, possible operating instructions 6 and a colorimetric scale 7 which allows the user to assess the final color of the mixture at the end of the reaction.

The kit can be used just once or, in contrast, can comprise sufficient amounts of reactants to allow several small locks to be characterized.

In an alternative form (not illustrated), the instructions 6 are replaced by instructions which appear on the box 1. It can be the same for the calorimetric scale 7, which can appear in the instructions or on the box 1, indeed even on one of the containers 4 or 5, if appropriate.

In the example of FIG. 2, the extraction solution present in the container 2 already contains a mixture of urea and of thiourea but it will not be departing from the scope of the present invention if the extraction solution is prepared at the time of use by mixing urea and thiourea.

In order to bring the extraction solution with a small lock M in the tube 3 to a predefined temperature, use may be made of a heating device 9 represented diagrammatically in FIG. 3.

An example of a packaging device 20 put together in order to separately store the urea and thiourea solution 21 and the reducing agent 22 while allowing them to mix subsequently has been represented in FIG. 4.

The device 20 comprises, for example, a body 23 and a removable part 24 which is put together in order to be attached to the body 23, for example by screwing or snapping. The removable part 24 comprises a base 25 provided with a leaktight annular lip 26 which is applied to the body 23 and with a rim 27 which acts as support for a seal 28 which makes it possible to isolate the reducing agent 22 from the urea and thiourea solution 21 during the storage.

The removable part 24 also comprises a top part 29 on which the user can press in order to break the seal 28 and to bring about the expulsion of the reducing agent 22 into the body 23 and the mixing thereof with the urea and thiourea solution 21.

The top part 29 comprises, in the example illustrated, in its central portion, an indentation 30, preferably pointed in shape, which makes it possible to perforate the seal 28. The latter is formed, for example, of a film heat-sealed at its periphery to the rim 27. The top part 29 is, for example, fixed by snapping or heat-sealing to the base part 25 after inserting the reducing agent 22.

In order to use the device represented in FIG. 4, the user can remove the removable part 24 and can introduce the small lock into the body 23, so as to expose it to the extraction solution 21. The body 23 can be placed in a heating device, as illustrated in FIG. 3. Once the extraction operation is complete, the user can put the removable part 24 back in place on the body 23 and can press on the top part 29 to cause the seal 28 to break and the reducing agent 22 to be added to the extraction solution 21.

Of course, other packaging devices can also be used.

### EXAMPLES

All the tests described below were carried out using the following keratinous fibers:

(For the purposes of clarity, the following coding symbols were adopted, N: natural, D: dyed, B: bleached, AT: alkaline hair straightening treatment, TT: thiol hair straightening treatment).

Caucasian Hair Untreated with a Hair Straightening Agent:

N: Natural hairs

B: Bleached hairs

D: Dyed hairs

Caucasian Hair Treated with a Hair Straightening Agent:

Alkaline Technology (AT):

GAT: Alkaline hair straightening Treatment based on Guanidine carbonate.

SAT: Alkaline hair straightening Treatment based on Sodium hydroxide

Thiol Technology (TT):

TT=Thiol hair straightening Treatment based on thioglycolic acid

Extraction Solution

The reducing solution, used in a proportion of a volume of 5 ml per test, is prepared from the following compounds at the specific concentrations:

Urea (7M) sold by Biorad, reference: 1610730,

Thiourea (2M) sold by Fluka, reference: 88810,

DTT (Dithiothreitol) (0.05M) sold by Sigma, reference: D-9779,

TRIS (0.05M) sold by Aldrich, reference: T8,760-2,

Triton X (0.1%) sold by Sigma, reference: T-8787,

Milli-Q water.

Detection Method

As regards the detection method used to characterized the dissolved proteins, it is a colorimetric method based on the Bradford method (Bradford, Anal. Biochem., 72, 248, 1976).

More particularly, the test used is the Bio-Rad Protein® test, sold by Bio-Rad, which uses Coomassie Blue® as colorimetric indicator.

The calibrating protein used is that recommended for the test, namely the bovine serum albumin (BSA) protein sold by Calbiochem under the commercial reference 12659.

### Example 1

The extraction procedure followed for all the tests is as follows:

A small lock of hair fibers (corresponding to approximately 50 mg) is cut, with scissors, into sections with a length of 2 to 3 mm which are brought into contact with 5 ml of the extraction solution described above. This contacting operation is carried out either at ambient temperature for 18 h or at a temperature of 100° C., provided by heating the extraction medium on a waterbath, for 15 minutes. The combined mixture is kept stirred using a magnetic bar throughout the extraction.

On conclusion of the extraction, the proteins in the supernatant are assayed using the Bio-Rad Protein Assay® commercial colorimetric kit.

To do this, 30 μl of supernatant are added to 5 ml of the calorimetric Bradford reagent (diluted beforehand to ⅕^{th} and filtered according to the instructions specified in the Bio-Rad kit). The color is allowed to develop for a few minutes.

The results are read as follows:

if a color develops (ranging from brown towards blue), the hair has not been subjected to alkaline hair straightening,

if the color does not change or changes only very slightly, the hair has been subjected to alkaline hair straightening.

From a color chart drawn up beforehand as a function of concentrations of proteins obtained with various treatments to which hairs have been subjected beforehand, it is possible to read the result visually and to reply to the question of whether or not an alkaline hair straightening has been carried out for the hair tested.

From a standard range of proteins, it is possible to quantify the proteins in the extraction solutions and to calculate a level of extraction with respect to the weight of hair used. The results appear in the tables below. The results are expressed therein in g of proteins extracted per 100 g of dry hairs and with reference to BSA.

The results obtained are presented in Tables I to III below.

TABLE I
Hairs untreated with a hair straightening agent

| | | 18 h | 15 min |
|---|---|---|---|
| | | 40° C. | 100° C. |
| | | % Extract | % Extract |
| NH | Natural hairs - control | 13.7 | 16.0 |
| DH | Dyed hairs | 16.6 | 15.5 |
| BH | Bleached hairs | 20.8 | 15.9 |

TABLE II
Hairs straightened with a thiol technology

| | | 18 h | 15 min |
|---|---|---|---|
| | | 40° C. | 100° C. |
| | | % Extract | % Extract |
| NH + TT | Natural hairs - straightened with | 11.0 | 12.3 |
| | thioglycolic acid | | |
| DH + TT | Dyed hairs straightened with | 15.2 | 12.3 |
| | thioglycolic acid | | |
| BH + TT | Bleached hairs straightened with | 15.6 | 15.0 |
| | thioglycolic acid | | |

TABLE II
Hairs straightened with alkaline technology

| | | 18 h | 15 min |
|---|---|---|---|
| | | 40° C. | 100° C. |
| | | % Extract | % Extract |
| NH + GAT | Natural hairs + straightened with guanidine | 0.0 | 0.2 |
| | carbonate | | |
| DH + GAT | Dyed hair + straightened with guanidine carbonate | 0.6 | 0.6 |
| BH + GAT | Bleached hair + straightened with guanidine | 3.0 | 2.6 |
| | carbonate | | |
| NH + SAT | Natural hair + straightened with sodium hydroxide | 0.4 | 0.5 |
| DH + SAT | Dyed hair + straightened with sodium hydroxide | 0.7 | 1.2 |
| BH + SAT | Bleached hair + straightened with sodium hydroxide | 4.4 | 3.1 |

It is noted that the hairs treated beforehand by an alkaline technology are immediately pinpointed by the lack of development of the color (remain brown or become slightly green) during the colorimetric test, which is reflected by a very low extraction of proteins, whereas the keratinous fibers which have not been subjected to an alkaline hair straightening treatment develop a blue color in combination with much higher levels of extraction of the proteins.

### Example 2

The preceding tests were repeated on small locks of hairs taken directly from the head. The results are presented in Table IV below:

TABLE IV

| Coding | Treatment | % Extract |
|---|---|---|
| A | Thiol smoothing | 11.4 |
| B | Guanidine carbonate hair straightening | 0.3 |
| C | Thiol smoothing | 18.5 |
| D | Thiol smoothing | 14.1 |
| E | Guanidine carbonate hair straightening | 0.3 |
| F | Thiol smoothing | 20.6 |

The preceding results are confirmed.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A process for detecting previous treatment of keratinous fibers with alkaline technology for hair straightening or smoothing, the process comprising:
bringing the keratinous fibers into contact with an extraction solution under conditions effective to cause dissolution of keratin proteins; and
conducting a qualitative and/or quantitative assay of the keratin proteins dissolved in the extraction solution;
wherein:
the extraction solution comprises a mixture of at least one of urea, thiourea and derivatives thereof, with at least one reducing agent; and
the at least one reducing agent is free from thiourea.

2. The process according to claim 1, wherein conducting the qualitative and/or quantitative assay comprises conducting a qualitative assay of proteins dissolved in the extraction solution.

3. The process according to claim 1, wherein the extraction solution comprises urea and thiourea in a urea:thiourea ratio of from about 5:1 to about 1:2.

4. The process according to claim 3, wherein the extraction solution comprises urea and thiourea in a urea:thiourea ratio of from about 4:1 to about 2:1.

5. The process according to claim 3, wherein the mixture is present in the extraction solution in an amount of from about 20 to about 80 percent by weight relative to a total weight of the extraction solution.

6. The process according to claim 5, wherein the mixture is present in the extraction solution in an amount of from about 30 to about 70 percent by weight relative to a total weight of the extraction solution.

7. The process according to claim 1, wherein the extraction solution comprises at least one thiol-type reducing agent.

8. The process according to claim 1, wherein the at least one reducing agent comprises at least one member selected from the group consisting of: thioglycolic acid, thioglycolic acid esters, thioglycolic acid amide derivatives, thiolactic acid, thiolactic acid esters, thiolactic acid amide derivatives, cysteamine, C₁-C₄ acylated derivatives of cysteamine, cysteine, N-acetylcysteine, thiomalic acid, pantetheine, 2,3-dimercaptosuccinic acid, sulphites of alkali metals or alkaline earth metals, bisulphites of alkali metals or alkaline earth metals, N-(mercaptoalkyl)-co-hydroxyalkylamides, N-monoalkyl-4-mercaptobutyramides, N,N-dialkyl-4-mercaptobutyramides, aminomercaptoalkylamides, derivatives of N-(mercaptoalkyl)succinamic acids, derivatives of N-(mercaptoalkyl)succinamides, alkylaminomercaptoalkylamides, an azeotropic mixture of 2-hydroxypropyl thioglycolate and of 2-hydroxy-1-methylethyl thioglycolate, mercaptoalkylaminoamides, N-mercaptoalkylalkanediamides and formamidinesulphinic acid derivatives.

9. The process according to claim 1, wherein the at least one reducing agent comprises at least one member selected from the group consisting of: glyceryl monothioglycolate, N-acetylcysteamine and N-propionylcysteamine.

10. The process according to claim 1, wherein the at least one reducing agent comprises at least one member selected from the group consisting of 2-mercaptoethanol, dithiothreitol (DTT), thioglycolic acid, and derivatives thereof.

11. The process according to claim 1, wherein the at least one reducing agent is present in the extraction solution in an amount of from about 0.01 to about 2 M.

12. The process according to claim 1, wherein bringing the keratinous fibers into contact with the extraction solution comprises employing from about 0.2 to about 100 mg of the keratinous fibers per 1 ml of the extraction solution.

13. The process according to claim 1, wherein bringing the keratinous fibers into contact with the extraction solution comprises immersing the keratinous fibers in the extraction solution and heating the combined keratinous fibers and extraction solution to a temperature of at least about 50° C.

14. The process according to claim 1, wherein conducting the qualitative and/or quantitative assay comprises assaying proteins by a chemical, physical, physiochemical or immunological assaying method.

15. The process according to claim 1, wherein conducting the qualitative and/or quantitative assay comprises bringing at least a portion of the extraction solution into contact with at least one compound capable of reacting with the keratin proteins and providing a calorimetric indication of the reaction.

16. A kit for characterizing a protein state of keratinous fibers, comprising:
a solution comprising at least one of urea, thiourea, and derivatives thereof;
at least one reducing agent, the reducing agent being free from thiourea; and
at least one compound capable of functioning as a colorimetric indicator for qualitatively and/or quantitatively assaying extracted keratin proteins.

17. The kit according to claim 16, wherein characterizing a protein state of keratinous fibers comprises determining whether the keratinous fibers were previously subjected to a treatment with alkaline technology for hair straightening or smoothing.

18. The kit according to claim 16, wherein the at least one reducing agent comprises at least one thiol-type reducing agent.

19. The kit according to claim 16, wherein the at least one reducing agent comprises at least one member selected from the group consisting of: thioglycolic acid, thioglycolic acid esters, thioglycolic acid amide derivatives, thiolactic acid, thiolactic acid esters, thiolactic acid amide derivatives, cysteamine, C₁-C₄ acylated derivatives of cysteamine, cysteine, N-acetylcysteine, thiomalic acid, pantetheine, 2,3-dimercaptosuccinic acid, sulphites of alkali metals or alkaline earth metals, bisulphites of alkali metals or alkaline earth metals, N-(mercaptoalkyl)-ω-hydroxyalkylamides, N-monoalkyl-4-mercaptobutyramides, N,N-dialkyl-4-mercaptobutyramides, aminomercaptoalkylamides, derivatives of N-(mercaptoalkyl)succinamic acids, derivatives of N-(mercaptoalkyl)succinamides, alkylaminomercaptoalkylamides, an azeotropic mixture of 2-hydroxypropyl thioglycolate and of 2-hydroxy-1-methylethyl thioglycolate, mercaptoalkylaminoamides, N-mercaptoalkylalkanediamides and formamidinesulphinic acid derivatives.

20. The kit according to claim 16, wherein the at least one reducing agent comprises at least one member selected from the group consisting of: glyceryl monothioglycolate, N-acetylcysteamine and N-propionylcysteamine.

21. The kit according to claim 16, wherein the at least one reducing agent comprises at least one member selected from the group consisting of 2-mercaptoethanol, dithiothreitol (DTT), thioglycolic acid, and derivatives thereof.

22. The kit according to claim 16, wherein the solution and the at least one reducing agent are packaged in a single device.

23. The kit according to claim 16, wherein the solution and the at least one reducing agent are packaged in separate containers.

24. The kit according to claim 16, further comprising a container for receiving the solution, the at least one reducing agent and the keratinous fibers.

25. The kit according to claim 16, wherein the at least one compound is packaged in a container.

26. The kit according to claim 16, further comprising operating instructions.

27. The kit according to claim 16, further comprising at least one of a colorimetric control and a color chart for qualitatively and/or quantitatively assaying the keratin proteins by evaluating color intensity in test results.
